(19) United States
(12) Plant Patent Application Publication
(10) Pub. No.: US 2012/0131708 P1
(43) Pub. Date: May 24, 2012

(54) Pinus plant named ANSU HWANGKEUMSONG

(50) ;

(76) Inventor: Song Jong Heon, Bananri (KR)

(21) Appl. No.: 12/927,625

(22) Filed: Nov. 19, 2010

(51) Int. Cl. A01H 7/00

(52) U.S. Cl. PLT/213

(57) ABSTRACT

A new and distinctive variety of Pinus thunbergii Parl plant named ‘Ansu Hwangkeumsong’. The ‘Ansu Hwangkeumsong’ plant is particularly characterized by its yellow colored needles.

### LATIN NAME OF THE GENUS AND SPECIES OF THE PLANT CLAIMED

Pinus thunbergii Parl

### VARIETY DENOMONATION

Ansu Hwangkeumsong

### BACKGROUND OF THE INVENTION

The present invention relates to a new and distinct variety of a Pinus plant, and more specifically, to a new and distinct variety of a Pinus thunbergii Parl plant. The Pinus thunbergii Parl plant is also known as a Japanese black pine.

### BRIEF SUMMARY OF THE INVENTION

The present invention relates to a new and distinctive variety of Pinus thunbergii Parl plant named ‘Ansu Hwangkeumsong’, which means ‘Ansu golden-leaf pine’ in Korean. The ‘Ansu Hwangkeumsong’ plant is particularly characterized by needles with a yellow color.

### BRIEF DESCRIPTION OF THE PHOTOGRAPHS

The photographs found in the figures illustrate the overall appearance of the Pinus plant named ‘Ansu Hwangkeumsong’. The shown colors that are as accurate as possible with color reproductions. Colors in the figures may not exactly depict the color values cited in the detailed botanical description that accurately describes the color of the ‘Ansu Hwangkeumsong’ plant.

FIG. 1 depicts the ‘Ansu Hwangkeumsong’ plant one year after grafting.

FIG. 2 depicts the ‘Ansu Hwangkeumsong’ plant one year after grafting on the right hand side of the picture; and a Pinus densiflora var. oculus draconis one year after grafting on the left hand side of the picture.

FIG. 3 depicts the ‘Ansu Hwangkeumsong’ plant one year after grafting.

FIG. 4 depicts the ‘Ansu Hwangkeumsong’ plant four years after grafting.

FIG. 5 depicts the ‘Ansu Hwangkeumsong’ plant four years after grafting.

FIG. 6 depicts the ‘Ansu Hwangkeumsong’ plant four years after grafting.

FIG. 7 depicts the ‘Ansu Hwangkeumsong’ plant.

FIG. 8 depicts the ‘Ansu Hwangkeumsong’ plant five years after grafting.

FIG. 9 depicts the ‘Ansu Hwangkeumsong’ plant six years after grafting.

FIG. 10 depicts the ‘Ansu Hwangkeumsong’ plant in the form of a mini tree six years after grafting.

FIG. 11 depicts the ‘Ansu Hwangkeumsong’ plant in the form of a bonsai six years after grafting.

FIG. 12 depicts a fully grown ‘Ansu Hwangkeumsong’ plant ten years after grafting.

FIG. 13 depicts a fully grown ‘Ansu Hwangkeumsong’ plant fifteen years after grafting.

FIG. 14 depicts a fully grown ‘Ansu Hwangkeumsong’ plant fifteen years after grafting.

FIG. 15 depicts the ‘Ansu Hwangkeumsong’ plant in the form of mini trees.

FIGS. 16A-D depicts the ‘Ansu Hwangkeumsong’ plant color change in seasons.

### DETAILED DESCRIPTION OF THE PHOTOGRAPHS

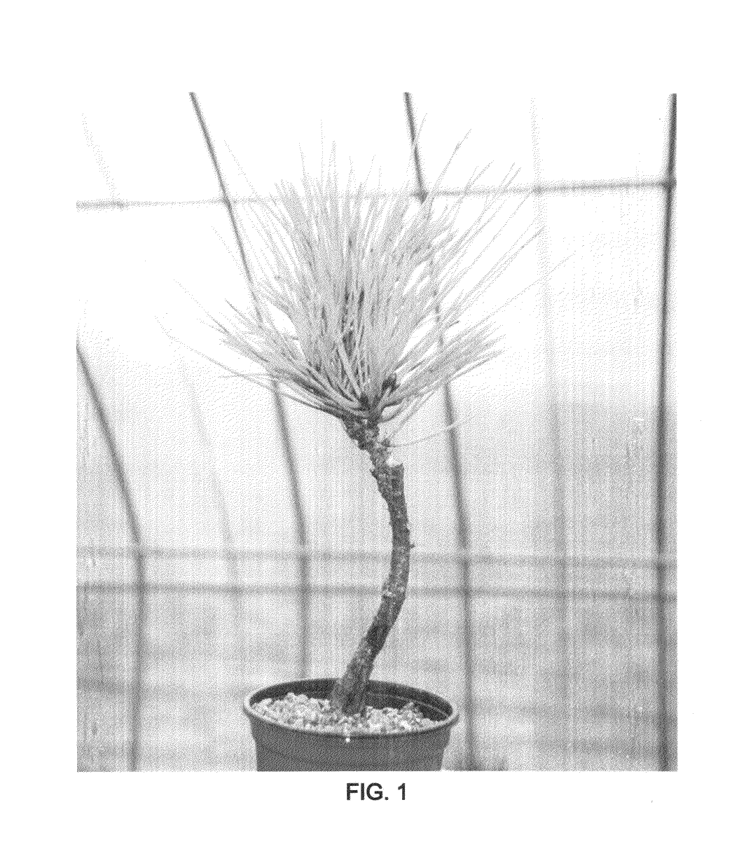

FIG. 1 illustrates the ‘Ansu Hwangkeumsong’ plant one year after grafting in a pot wherein the needles were sprouted from a bud after carrying out the pinching.

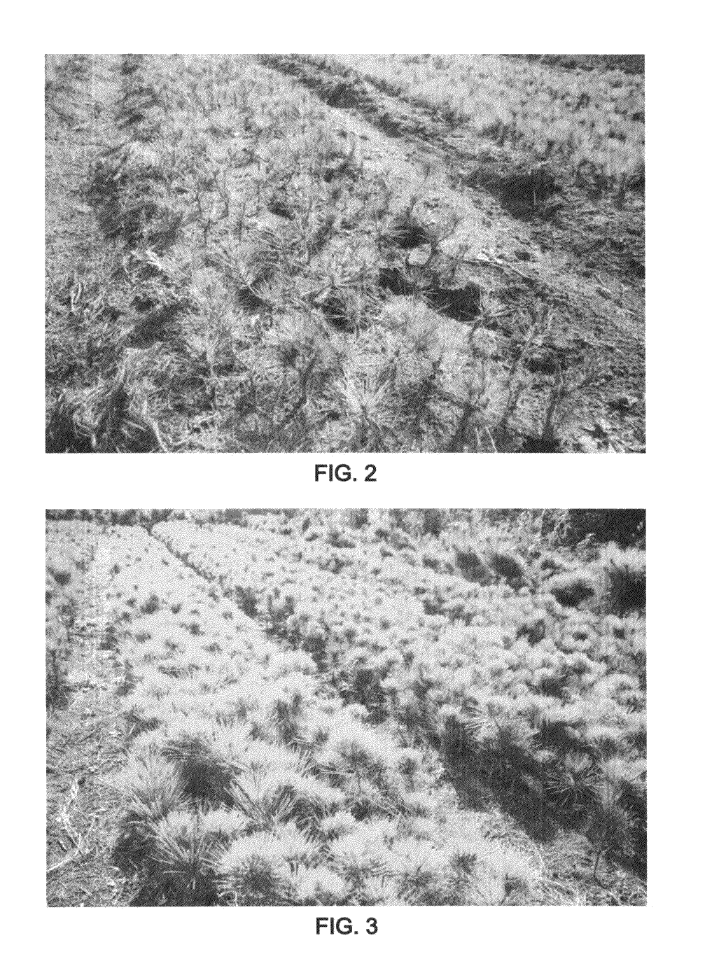

FIG. 2 depicts the ‘Ansu Hwangkeumsong’ plant one year after grafting on the right hand side of the picture; and a Pinus densilflora var. oculus draconis plant one year after grafting on the left hand side of the picture. The Pinus densilflora var. oculus draconis plant shows a yellow color near the basal end of the needle and a green color around its end of the needle. In contrast, the ‘Ansu Hwangkeumsong’ plant shows the whole needle being yellow.

FIG. 3 depicts the ‘Ansu Hwangkeumsong’ plant one year after grafting. Grafting was successfully carried out using 2-year-old black pine as a stock in Spring. It was found that 99% of the grafted variety remained characteristics of a mother tree (off-types did not occur). Some of the seedlings grown in this nursery field have been pinched, and others have not been pinched.

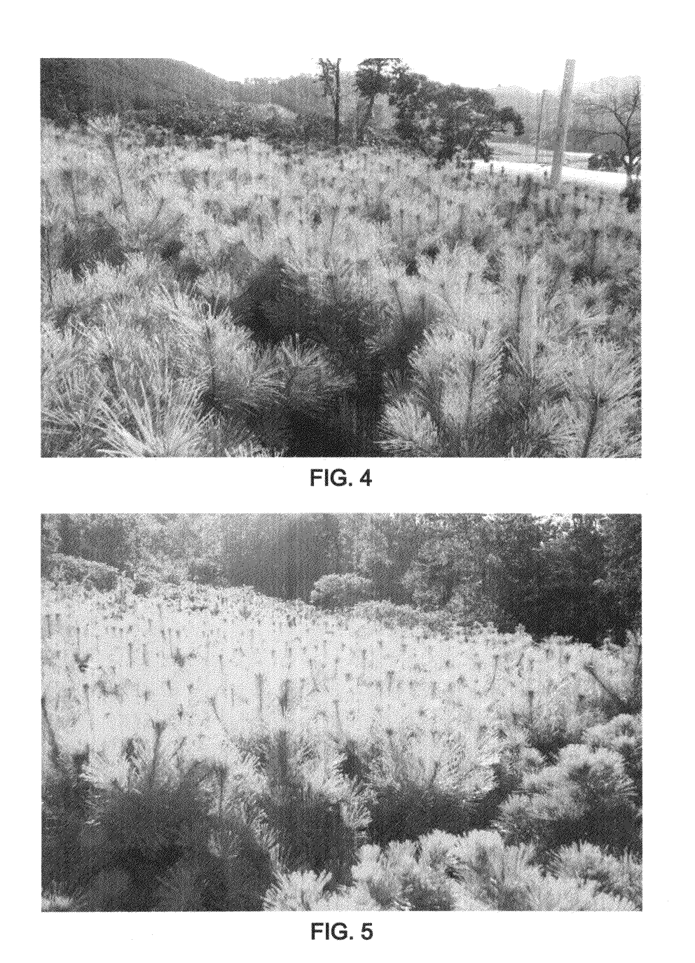

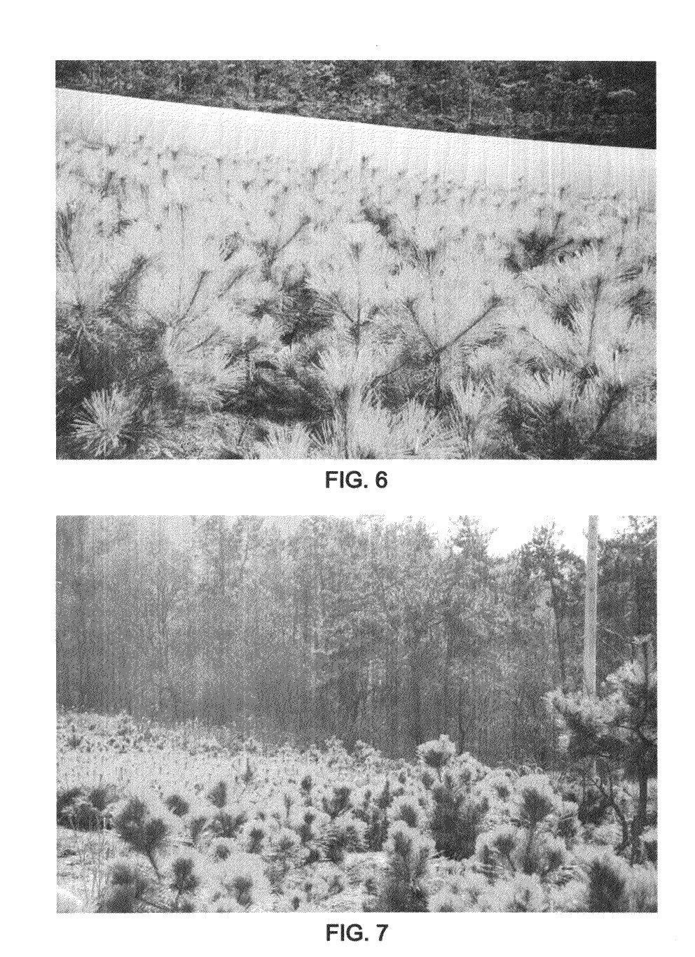

FIG. 4 depicts the ‘Ansu Hwangkeumsong’ plant four years after grafting. As shown the plant has a light yellow with a strong yellow tinge at the exterior part of the needles. FIG. 5 also depicts the ‘Ansu Hwangkeumsong’ plant four years after grafting. The photographs of FIGS. 4 and 5 were taken at the same site. FIG. 5 was taken a little over a month after the photograph from FIG. 4 was taken. As shown, the yellow color has been developed better in FIG. 5 when compared to FIG. 4. FIG. 6 also depicts the ‘Ansu Hwangkeumsong’ plant four years after grafting. The tree height is about 1.5 m. Since the growth potential is little inferior to the reference variety black pine, the average annual growth is about 50 cm.

FIG. 7 depicts the ‘Ansu Hwangkeumsong’ plant. The trees at the front are those pinched. The trees on the left in the middle of the photo are four years old. The tall trees in the background are indigenous black pine.

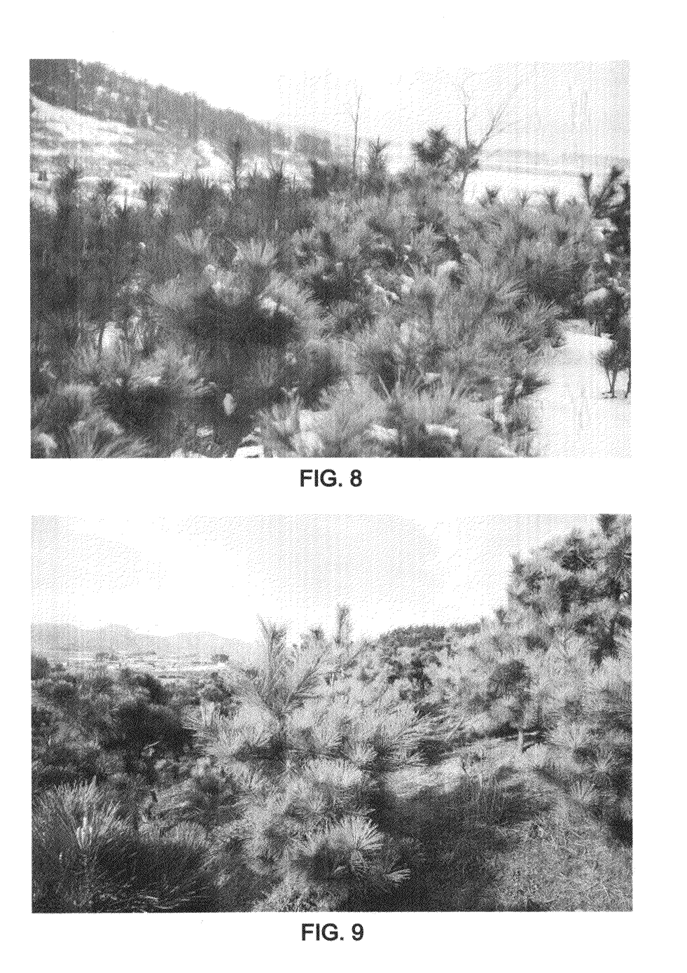

FIGS. 8 and 9 depict the ‘Ansu Hwangkeumsong’ plant five and six years after grafting, respectively. The trees on the left in the photos are the reference variety black pine.

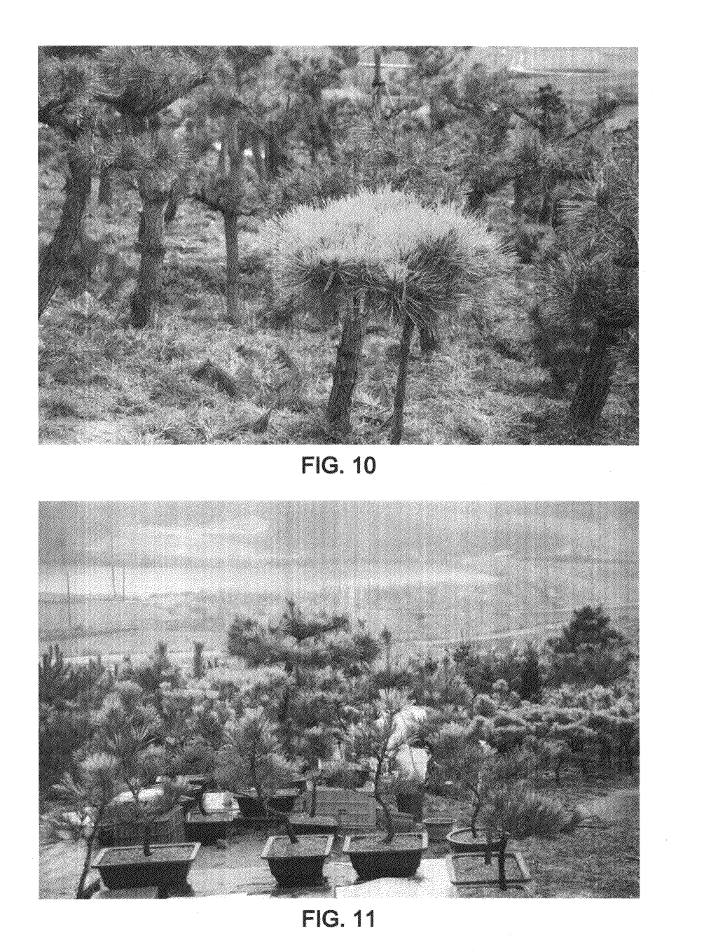

FIG. 10 also depicts the ‘Ansu Hwangkeumsong’ plant in the form of a mini tree six years after grafting. The mini tree was trained to have a height of 1 m. The trees in the background are the reference variety, black pine. FIG. 11 depicts the ‘Ansu Hwangkeumsong’ plant in the form of a bonsai six years after grafting.

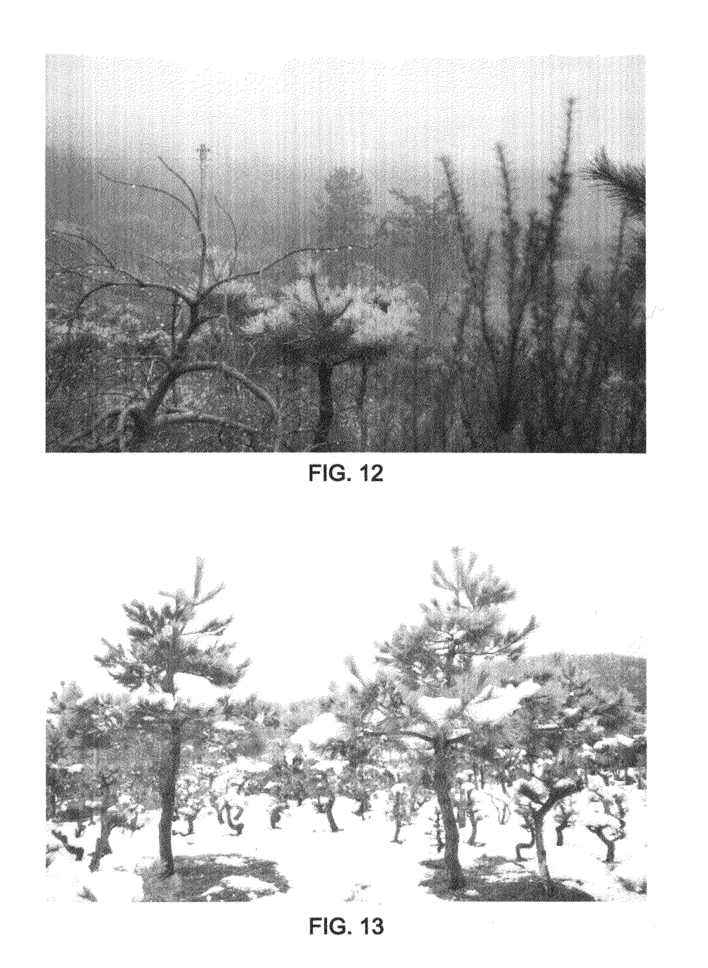

FIG. 12 depicts a fully grown ‘Ansu Hwangkeumsong’ plant ten years after grafting. The illustrated ‘Ansu Hwangkeumsong’ plant has a width of 2 m. Since the upper part of the tree receives a sufficient amount of sunlight, the yellow color is particularly well developed in the upper part. The tall trees far behind the ‘Ansu Hwangkeumsong’ plant are indigenous black pine trees.

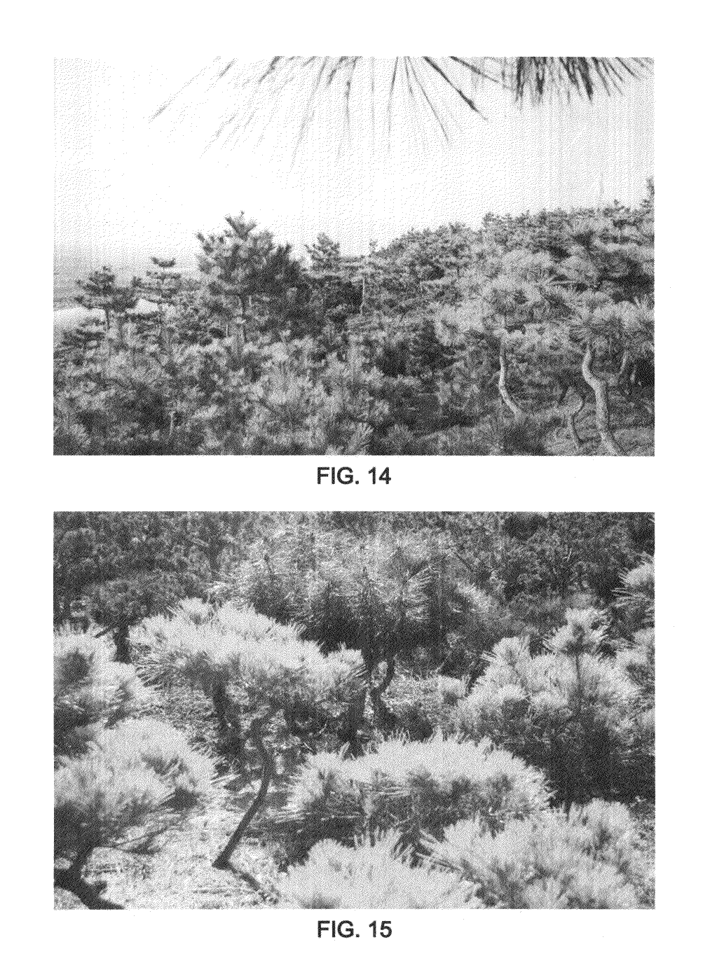

FIGS. 13 and 14 depict a fully grown ‘Ansu Hwangkeumsong’ plant fifteen years after grafting. The tree in FIG. 13 has a height of 5 m and a width of 4 m. The shapes of the trees shown in FIG. 14 have been trained by pruning, except the one slightly left in the middle of the photo (not pruned).

FIG. 15 depicts the ‘Ansu Hwangkeumsong’ plant in the form of mini trees. The mini trees have a height of about 1 m, trained by pinching and pruning. Trees in the rear part are general pine and black pine trees.

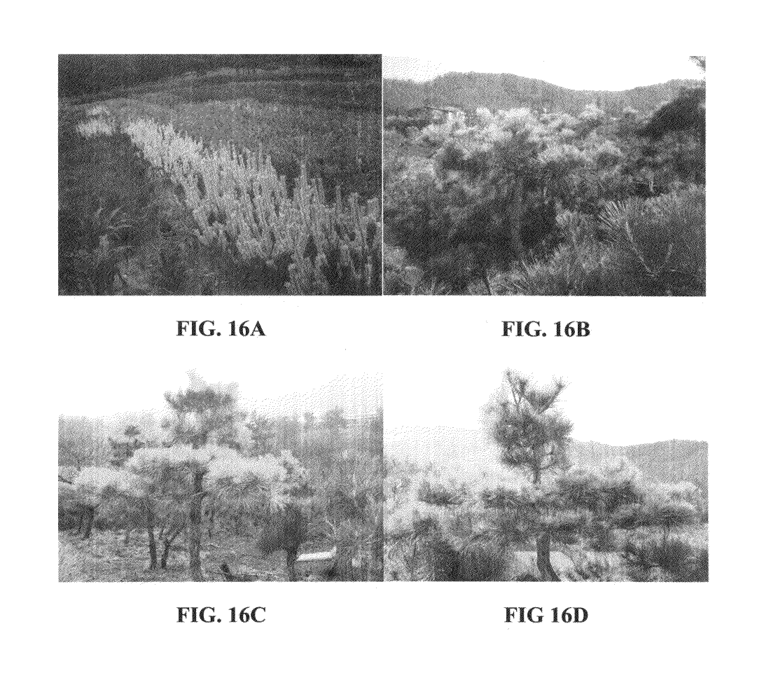

FIGS. 16A-D depict the ‘Ansu Hwangkeumsong’ plant color change in seasons described more fully in subsection section 2(1) in the Detailed Botanical Description below.

### DETAILED BOTANICAL DESCRIPTION

1. Breeding scheme

In 1995, the breeder (Song Jong Heon) of this plant variety discovered the variety, which had yellow needles in the side branches and shoots with a yellow-colored tip, among indigenous 3 year-old black pines in the breeder's tree farm (Ansu Tree farm, located in Jeollanam-do Yeonggwang-gun, Gunnam-myeon Bananri San 14-1, South Korea), and thus selected the same.

In 1996, a bud was taken from the selected variety as a scion and grafted to a stock that was a 4 year old black pine. Total 30 grafts were prepared.

In 1997, 5 graft seedlings were bred. Starting from these seedlings, 30 seedlings in 1998, 100 seedlings in 1999 and 1000 seedlings in 2000 were propagated via grafting, and evaluation on them was made in nursery field.

In 2009, about 30000 numbers of the present variety, ‘Ansu Hwangkeumsong’ plant are being cultivated. Those obtained at the beginning have grown into a tree with a height of 6 m and a width of 6 m, at present. All the trees of this new variety have remained with the intact parental variety's characteristics with uniformity and stability.

2. Characteristics of the variety
(1) Color (color changes with the seasons)

The color change in seasons can be observed in FIGS. 16A-D described below:

1) FIG. 16A illustrates ‘Ansu Hwangkeumsong’ plant from May-June (Spring): The middle part of FIG. 16A shows the ‘Ansu Hwangkeumsong’ plant in light green while the left side of FIG. 16A illustrates the reference variety in green. New pine needles sprout, with a yellow-green or green color with a yellow tint; and a stalk from a winter bud is golden-yellow.

2) FIG. 16B illustrates ‘Ansu Hwangkeumsong’ plant from July-August (Summer): Needles from new stalks become greener, however it is light and delicate green as compared to the reference variety, Pinus thunbergii Parl plant.

3) FIG. 16C illustrates ‘Ansu Hwangkeumsong’ plant from around the end of August (Fall), the needles begin to turn yellow from the tip thereof, and after the first frost of the season, the color of the needles is quickly changed to yellow all over the needles.

4) FIG. 16D illustrates ‘Ansu Hwangkeumsong’ plant from October-April (Fall-next Spring): Yellow needles, clearly distinguished from the reference variety. As shown in the upper part, needles from the primary bud are shown (pinching was not carried out). As shown in the lower part, needles from the secondary bud are shown (pinching was carried out in May).

(2) Biological characteristics 1) Owing to the similar basic biological characteristics inherent to Pinus thunbergii Parl plant. such as good environmental adaptability, growth of the present variety can be promoted by fertilizing and drainage control.

2) Desired tree shape can be easily obtained by pruning.

3) Despite of the color change in needles, any problems in growth do not occur.

(3) Cultivation techniques and environmental conditions

1) By pinching the primary bud in mid-May, deep yellow (golden) color is distinctively developed in the needles (2 to even 5 needles come out when the tree vigor is good) and stalks from the secondary bud, as compared to the golden-yellow color appeared in the needles from the primary bud, which is one of the most unique feature of this variety.

2) When the variety of this application is in good vigor and cultivated in the sufficient sunshine, the golden-yellow color can be developed better.

3) When the present variety is planted in a sunny spot, rather separated from other trees, the yellow color is developed better. The most vivid and healthy-looking yellow color in the needles can be obtained by pruning which provides good ventilation and sunlight. For developing more vivid color, pinching of buds is performed in mid-May, thus promoting development of the secondary bud.

4) On the contrary, in the shady place, the yellow color is not desirably developed. Moreover, in the heavy shade, the yellow color is not developed at all, in some cases. When planted densely, the yellow color is not vivid.

5) Although excess use of commercial fertilizers causes darkening and discoloration in the yellow color of the needles, a suitable amount of fertilizers may be helpful for fine growth and color development in the present variety.

6) When the soil is unproductive and no fertilizer is used, the whole body of each needle may become yellow, although the yellow color is rather dark.

7) The present variety should be propagated by grafting. The present variety can be suitably grown for a mini tree, pot gardening and Bonsai.

(4) Characteristic features in the growth of the present variety

1) In early May, the basal part of a winter bud starts to grow out with yellow tint.

2) In June, newly sprouted needles from winter buds have yellow-green color with yellow tint. The new needles are clean and bright at this stage.

3) In the summer, although the needles become greener, they are still much soft green as compared to the needles of the reference variety.

4) Around the end of August, from the tip of a newly sprouted needle the color becomes yellowish. Around the time when the first frost comes, the color quickly turns to yellow. When a secondary bud was developed by carrying out pinching (cutting out some end portion of a bud) at the time around early June, bright and vivid golden-yellow color can be obtained around the end of August in a robust tree.

5) From winter to the time when new needles start to grow in the next year, the yellow color is maintained.

6) When a secondary bud was developed by carrying out pinching at the time around early June, yellow color can be developed better.

7) The preceding characteristic features can be strongly appeared, only if good drainage, sufficient sunlight and soil with a small amount of fertilizer are provided. When grafted, the features are little bit weakened.

(5) Uniformity and stability of the present variety

1) From the first discovery of the mother tree, the characteristic features of the mother tree of the present variety have been descend to and maintained in all of the offspring which have been propagated by grafting.

2) Tree height

| Elapsed year after grafting | Average tree height(cm) |
|---|---|
| 1 | 15 |
| 5 | 150 |
| 10 | 300 |

(6) Characteristics distinctive from reference variety (Pinus thunbergii; Black pine)

1) The color of the needles (leaves) of the present variety is yellow.

| Color(RHS) | ‘Ansu Hwangkeumsong’ | Pinus thunbergii; Black |
|---|---|---|
| of needles | plant (Present variety) | pine (reference) |
| Fall-winter | Yellow Group 13A | Yellow-green |
| | | Group 152A |
| Spring | Yellow Group 10A | Green Group 137B |
| Summer | Green Group 143B | Green Group 137B |

2) The needles are shorter and thicker than those of the reference variety.

3) Growth, in particular growth in woody part thereof is rather slower that of the reference variety.

(7) Additional information which may help to distinguish the variety

1) Resistance to pests and diseases

Growth potential of the present variety is comparable with that of the reference variety, which has an excellent growth potential, in spite of the yellow needle color. However, when attacked by Spruce spider mite (Oligonychus ununguis), the appearance of a tree is easily affected as compared to the reference variety.

2) Physiological disorder

When planted in the soil containing lime, seedlings sometimes show reddish tinge at the tip or in the middle of the needles, especially during July-November. However, such disorder is not appeared in the trees with good vigor and mature trees.

3) Conditions suitable for cultivation of the present variety

the mean annual temperature suitable for growth: 10-16° C.

the mean monthly temperature suitable for growth: May, 16-19° C.; August, 23-27° C.; October, 11-19° C.; January, −6 to 7° C.

Soil: pH 4.5-6.8; sandy soil having good drainage

(8) Other information

When enough sunlight is given to robust branches of the present variety, the yellow color is always well developed.

In order to obtain fine golden-yellow color in a mature tree of this variety, it is considered that the tree shape is needed to be trained so that every needle in the tree can receive enough sunlight throughout the tree.

What is claimed is:

1. A new and distinctive variety of Pinus thunbergii Parl plant named ‘Ansu Hwangkeumsong’ as illustrated and described.
